(19) United States
(12) Patent Application Publication
(10) Pub. No.: US 2023/0414558 A1
(43) Pub. Date: Dec. 28, 2023

(54) PLECTRANTHUS AMBOINICUS EXTRACT FOR USE IN ALLEVIATION OF RADIATION-INDUCED SKIN DISORDERS

(71) Applicant: Oneness Biotech Co., Ltd., Taipei City (TW)

(72) Inventors: Kung-Ming LU, Taipei City (TW); Shyi-Gen CHEN, Taipei City (TW); Jui-Ching CHEN, Taipei City (TW)

(21) Appl. No.: 18/342,313

(22) Filed: Jun. 27, 2023

Related U.S. Application Data
(60) Provisional application No. 63/355,995, filed on Jun. 27, 2022.

(51) Int. Cl. A61K 31/352; A61K 36/53; A61K 31/704; A61K 36/23; A61K 9/06; A61P 17/00; A61P 29/00; A61K 45/06

(52) U.S. Cl. CPC A61K 31/352; A61K 36/53; A61K 31/704; A61K 36/23; A61K 9/06; A61P 17/00; A61P 29/00; A61K 45/06; A61K 2236/31; A61K 2236/53

(57) ABSTRACT

A method for alleviating radiation-induced skin disorder (e.g., radiation dermatitis), comprising administering to a subject in need thereof an effective amount of a pharmaceutical composition comprising salvigenin, and optionally cirsimaritin, rosmarinic acid, carvacrol, or a combination thereof.

### CROSS-REFERENCE TO RELATED APPLICATIONS

This application claims the benefit of priority to U.S. Provisional Patent Application No. 63/355,995, filed Jun. 27, 2022, the disclosures of which are incorporated herein by reference in the entirety.

### BACKGROUND OF THE INVENTION

Plectranthus amboinicus (also known previously or alternatively as Coleus amboinicus Lour., Coleus aromaticus Benth., Coleus aromaticus auct., Plectranthus aromaticus Roxb., Plectranthus aromaticus Benth., and Plectranthus amboinicus (Lour.) Spreng.), is a perennial medicinal herb of the Lamiaceae (also known as Labiatae) family native to Southern and Eastern Africa. Plectranthus amboinicus is also known as patchouli, Cuban oregano, Indian borage, Indian mint, Mexican mint, Mexican oregano, country borage, and Spanish thyme.

Centella asiatica (also known previously or alternatively as Centella asiatica Urban, Centella asiatica (L.) Urban, Hydrocotyle asiatica L., and Trisanthus cochinchinensis Lour.) is a perennial medicinal plant of the family Mackinlayaceae or subfamily Mackinlayoideae of the Apiaceae (also known as Umbelliferae) family native to Asia, Africa, and South America. Centella asiatica is also known as European water-marvel, gotu kola, Kola, pennywort, Indian pennywort, marsh pennywort, pennyweed, Indian ginseng, Horse-hoof grass, Pegaga, Mandookaparni, Tiger herbal, Spadeleaf, or Tono. Extracts of Centella asiatica generally comprise two major compounds: asiaticoside and madecassic acid.

### SUMMARY OF THE INVENTION

The present disclosure is based, at least in part, on the unexpected discoveries that compositions comprising salvigenin, and optionally cirsimaritin, rosmarinic acid, carvacrol, or a combination thereof (e.g., Plectranthus amboinicus (PA) extract, optionally in combination with Centella asiatica (CA) extract) successfully reduced disease scores of radiation dermatitis and the therapeutic effects were better than a corticosteroid control. Accordingly, the composition disclosed herein are expected to exhibit superior efficacy in alleviating radiation dermatitis.

In some aspects, provided herein is a method for alleviating radiation-induced skin disorders (e.g., radiation dermatitis, radiation-induced mucositis, radiation-induced lichen planus, radiation-induced vaginitis, radiation-induced skin inflammation, radiation-induced skin damage, or a combination thereof), the method comprising administering to a subject in need thereof an effective amount of a composition comprising salvigenin and a carrier. In some embodiments, the composition may be a pharmaceutical composition, which may further comprise a pharmaceutically acceptable carrier. The composition disclosed herein may further comprise cirsimaritin, rosmarinic acid, carvacrol, or a combination thereof.

In some embodiments, the composition comprises a Plectranthus Amboinicus (PA) extract. In some examples, the PA extract can be prepared by a process comprising: (i) mixing a part of PA (e.g., an above-grant part) with an extracting solution to produce a first PA extract, (ii) filtrating and concentrating the first PA extract to produce a concentrated PA extract; (iii) contacting the concentrated PA extract onto a hydrophobic interaction chromatography resin, and (iv) eluting the column with a eluent solution to product the PA extract. In some examples, the extracting solution comprises a solvent having a polarity index of about 2.9 to 6.6; optionally wherein the extracting solution is acetone, butyl methyl ether, ethanol, ethyl acetate, isopropyl alcohol, methanol, or a mixture thereof. In some examples, the eluent solution comprises a solvent having a polarity index of about 2.1-5.4; optionally wherein the eluent solution comprises a mixture of at least two solvents selected from the group consisting of acetone, ethanol, ethyl acetate, and hexane.

In some embodiments, the composition further comprises asiaticoside. For example, the composition further comprises a Centella asiatica (CA) extract, which comprises the asiaticoside.

In some embodiments, the subject for treatment by any of the methods disclosed herein is a human patient having any of the radiation-induced skin disorder (e.g., radiation dermatitis, radiation-induced mucositis, radiation-induced lichen planus, radiation-induced vaginitis, radiation-induced skin inflammation, radiation-induced skin damage, or a combination thereof). In some embodiments, the composition is a topical formulation (e.g., in a suitable form such as a cream, gel, dressing, spray formulation, ointment, paste, patch, or lotion). In some embodiments, the composition is applied to a site where the skin disorder occurs. In some instances, the composition is administered to the subject (e.g., a human cancer patient) who is receiving a radiotherapy.

In some embodiments, any of the compositions disclosed herein may be applied to the subject at least once per day (e.g., up to 6 times per day). Alternatively, the composition may be applied to the subject once every a few days (e.g., up to 3 days).

Also within the scope of the present disclosures are the composition as disclosed herein for use in alleviating radiation-induced skin disorders such as those disclosed herein in a subject in need thereof, and the composition for use in manufacturing a medicament for the intended medical uses.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic illustration of an experimental design for a mouse radiation study to explore efficacy of ON101 and PA-F4 cream in treating radiation dermatitis and to determine biomarkers of radiation exposure.

FIG. 2 is a diagram showing reduction of disease scores by ON101 and PA-F4 creams in comparison with a corticosteroid cream control using a radiation dermatitis mouse model. *P<0.05, **P<0.01, ***P<0.001.

FIG. 3 is a diagram showing thickness of epidermal of skin tissues from untreated mice or mice treated with placebo cream, PA-F4, or ON101. *P<0.05, **P<0.01, ***P<0.001.

FIG. 4 is a diagram showing fibrosis contents in skin tissues from untreated mice or mice treated with placebo cream, PA-F4, or ON101. *P<0.05, **P<0.01, ***P<0.001.

FIGS. 5A-5C include diagrams showing levels of NLRP3 (FIG. 5A), IL1β (FIG. 5B), and γ-H2AX (FIG. 5C) of skin tissues from untreated mice or mice treated with placebo cream, PA-F4, or ON101. Protein express levels are represented by Q-Score values. *P<0.05, **P<0.01, ***P<0.001.

FIG. 6 is a diagram showing differentiated expression of various protein markers as indicated in skin tissues of untreated mice and mice treated by ON101. ***P<0.001.

FIGS. 7A-7D include diagrams showing protein levels of skin tissue samples from untreated mice or mice treated with placebo cream, PA-F4, or ON101. FIG. 7A: Westernblot for measuring levels of Keap1, p62, Nrf2, NF-κB, γ-H2AX, P53, and GAPDH. FIG. 7B: Westernblot for measuring levels of nuclear and cytoplasmic Nrf2. FIG. 7C: levels of Nrf2 in nucleus. FIG. 7D: levels of Nrf2 in cytoplasm.

### DETAILED DESCRIPTION OF THE INVENTION

Radiation can induce skin disorders. Radiation dermatitis, also known as X-ray dermatitis, is a common side effect of radiotherapy. Radiation dermatitis involves damage of skin cells caused by radiation. Typical symptoms include redness, skin peeling, and ulceration. It usually begins to resolve after treatment ends. In some cases, it can appear years after radiotherapy has finished. In patients having radiation dermatitis, skin cell damage can occur at the deep layer of skin tissues without obvious open surface wound. Instead of necrosis or apoptosis, the cell structure around the damage regions may be altered.

It is reported herein that, unexpectedly, an PA extract (e.g., comprising salvigenin and optionally cirsimaritin, rosmarinic acid, carvacrol, or a combination thereof), either alone or in combination with a CA extract (e.g., comprising asiaticoside) showed high therapeutic effects in treating radiation dermatitis as observed in an animal model. Accordingly, provided herein are pharmaceutical compositions comprising Plectranthus amboinicus extract or active agents thereof, and optionally Centella asiatica extract or active agents thereof, for use in alleviation of radiation dermatitis.

### I. Compositions

The instant disclosure herein provides a composition such as a pharmaceutical composition for alleviating radiation-induced skin disorders, e.g., radiation dermatitis, radiation-induced mucositis, radiation-induced lichen planus, radiation-induced vaginitis, radiation-induced skin inflammation, radiation-induced skin damage, or a combination thereof. The compositions disclosed herein may comprise a PA extract or one or more active agents contained therein (e.g., salvigenin, and optionally cirsimaritin, rosmarinic acid, carvacrol, or a combination thereof). In some embodiments the pharmaceutical composition may further comprise a CA extract or one or more active agents contained therein (e.g., asiaticoside).

(i) Active Agents

The active agent in the composition described herein comprises salvigenin. In some embodiments, the active agent may further comprise cirsimaritin, rosmarinic acid, carvacrol, or a combination thereof. In some instances, the composition comprises a PA extract, which comprises salvigenin and optionally one or more of cirsimaritin, rosmarinic acid, and carvacrol.

Plectranthus amboinicus Extract

A PA extract refers to an extract obtained from PA plant using one or more suitable solvents. In some examples, the PA extract is prepared using an above-ground part of the PA plant. In some instances, at least one of the solvents used for preparing the extract has a polarity index lower than 7 (e.g., less than 5). See, e.g., U.S. Pat. No. 10,758,584, the relevant disclosures of which are incorporated by reference for the subject matter and purpose referenced herein. As used herein, a solvent refers to a substance or a mixture of substances that dissolves another to form a solution. A PA extract as described herein may be prepared using one solvent. The solvent used in each extracting step for preparing the extracts described herein (including both PA and CA extracts) may be a single solvent. Alternatively, it can be a mixture of two or more solvents.

The PA extract described herein may comprise terpenoids (e.g., monoterpenoids, diterpenoids, triterpenoids, and/or sesquiterpenoids), flavonoids, phenolics, essential oils, or a combination thereof. The PA extract for making the pharmaceutical compositions disclosed herein may comprise salvigenin, and optionally one or more of cirsimaritin, rosmarinic acid, and carvacrol.

The PA extract described herein may be prepared by extracting a whole PA plant or a part thereof (e.g., an above-ground part) with one or more suitable solvents to produce a solution and then drying the solution to produce the PA extract. Since the PA extract comprises flavonoids, terpenoids (e.g., monoterpenoids, diterpenoids, triterpenoids, and/or sesquiterpenoids), phenolics, or essential oils, which are non-polar molecules, at least one of the extracting solvents may have a relatively low polarity (e.g., having a polarity index lower than 7) to facilitate dissolution of the non-polar molecules. “Extracting” can be performed by either contacting the PA material directly with a suitable solvent or by eluting active components of PA from resins on which the active components are attached.

In some examples, a solvent having a polarity index lower than 7 can be used for extracting the active components from PA to produce the PA extract. Such a solvent can be ethyl acetate, methyl acetate, propanol, butanol, or chloroform. Alternatively, the solvent can be a mixture of one or more solvents having different polarity index. Examples include, but are not limited to, a mixture of ethanol and ethyl acetate, ethyl acetate and butanol, ethanol and propanol, methyl acetate and butanol.

In some embodiments, the PA extract may be prepared by a process involving the use of one solvent, such as a solvent having a polarity index lower than 7 (e.g., <about 6.5, <about 6.0; <about 5.5, <about 5.0, <4.9, <4.8, <4.7, <4.6, or <4.5). Examples include, but are not limited to, methanol, ethanol, acetone, ethyl acetate, butanol, dichloromethane, or a combination thereof.

PA materials, which can be a whole PA or a part thereof (e.g., an above-ground part such as leaves), can be prepared by routine methodology. The PA material can be a fresh plant or a part thereof. Alternatively, the PA material can be in dried form. The PA can optionally be dried to form powders, which can be used as the PA material for preparing the PA extract.

Any of the PA materials as described herein can be extracted, one or more times, by a suitable solvent to produce a crude extract. The solvent for use in preparing the crude extract may be a high-polarity solvent, for example, having a polarity index above 5 and preferably below 7 (e.g., >5.2; >5.5, >5.8, >6 or above and preferably below 7). Examples include, but are not limited to, ethanol, acetone, methanol, water, or a combination thereof. If necessary, the crude extract can be concentrated by a conventional method to produce a concentrated crude extract.

The crude extract can then be brought in contact with a suitable resin (e.g., a non-ionic absorbent resin) under suitable conditions that allow for binding of active components in the crude extract onto the resin. Exemplary resins for use in preparing the Plectranthus amboinicus extract include, but are not limited to, DIAION® HP20, DIAION® HP20SS, Sepabeads® SP207, Amberlite™ XAD-2, or Amberlite™ XAD-4.

Afterwards, the resin can be washed one or more times and eluted with a suitable solvent, for example, a solvent having a polarity index lower than 7, to produce a PA extract, which can then be dried by a conventional method (e.g., freeze-drying, spray-drying, or concentration drying) to produce dried the PA extract, which can be in semisolid or paste form.

In some examples, the resin absorption step can be performed by mixing the crude extract with the resin in a container. In other embodiments, the resin separation step can be performed by a chromatography column setting.

In one example, an extract of PA can be prepared as follows. An overground part of PA (about 1.5 g), including leaves and/or stems, can be collected and extracted with a solvent having a polarity less than 7 (e.g., methanol, ethanol, acetone, ethyl acetate, butanol, dichloromethane, or a combination thereof) at room temperature for 30 min to 6 hours. Alternatively, this extraction process can be carried out at a temperature of about 50 to 80° C. The resultant crude extract can be directly loaded to a non-ionic adsorbent resin column and eluted by a solvent having a polarity less than 6 (e.g., ethanol, ethyl acetate, butanol, dichloromethane, hexane, toluene, or a combination thereof). The eluted components can be collected and purified by extraction with a solvent having a polarity less than 6 (e.g., those described herein). The resultant filtrate can be collected to produce a PA extract.

Centella asiatica Extract

In some embodiments, the composition such as the pharmaceutical composition disclosed herein may further comprise asiaticoside. In some instances, the composition further comprises a CA extract, which may comprise the asiaticoside compound. The CA extract as described herein refers to an extract obtained from whole CA plants or a part thereof. The CA extract may comprise asiaticoside, and optionally madecassic acid.

The CA extract can be prepared following a conventional method, for example, those described in U.S. Pat. Nos. 5,834,437, 6,417,349, 6,475,536, and 6,267,996, CN 1313124, CN 1089497, and CN 1194154. Below is an example.

CA materials can be prepared via routine practice. Such materials can be fresh CA plant or a part thereof, or dried CA. The CA materials can be extracted with a suitable solvent such as water, ethanol, or a mixture thereof, to produce a crude extract. The crude extract, which can be optionally concentrated, can be either mixed with a suitable resin or loaded onto a column packed with the resin. After being washed for one or more times, the resin can be eluted with a suitable solvent. The resultant eluent can be concentrated to form a paste, which can be dried by a conventional method, for example, vacuum dry, to produce powders of the CA extract. When necessary, the CA powder can be grinded through a mesh (e.g., a No. 100 mesh).

An extract of CA can be prepared by the same or similar process as described above for making the PA extract. The PA Extract and/or CA Extract may be concentrated using a pressure-reducing rotary evaporator.

(ii) Pharmaceutical Compositions

Any of the active compounds (e.g., salvigenin, and optionally one or more of cirsimaritin, rosmarinic acid, carvacrol, and/or asiaticoside), the PA extract, optionally in combination with the CA extract as disclosed herein can be mixed with a suitable carrier (e.g., a pharmaceutically acceptable carrier) to form a composition (e.g., a pharmaceutical composition) for use in alleviating radiation-induced skin disorders such as radiation dermatitis, radiation-induced mucositis, radiation-induced lichen planus, radiation-induced vaginitis, radiation-induced skin inflammation, radiation-induced skin damage, or a combination thereof. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The compositions such as pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the active agent, which can be prepared by methods known in the art or such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active agent may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the active agents, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The compositions such as the pharmaceutical composition to be used for in vivo administration must be sterile. This is readily accomplished by, for example, filtration through sterile filtration membranes. The compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing the active agents with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Topical Formulations

In some embodiments, the pharmaceutical composition disclosed herein is a topical formulation. Such a topical formulation may comprise any of the PA extract disclosed herein or one or more active agents thereof (e.g., salvigenin, and optionally in combination one or more of cirsimaritin, rosmarinic acid, carvacrol). In some instances, the topical formulation may further comprise a CA extract as disclosed herein or one or more active agents thereof (e.g., asiaticoside). In some examples, the topical formulation described herein may comprises salvigenin in an amount ranging from about 0.0001% to about 0.5% (w/w), and optionally asiaticoside, which may be in an amount ranging from about 0.05% to about 5% (w/w). In some embodiments, the topical formulation comprises the Plectranthus amboinicus extract, the Centella asiatica extract, or both in an amount of about 0.1-30% (w/w).

As used herein, the term “about” intends to a strict numerical boundary to the specified parameters (including both the upper and lower limits). A skilled person in the art would have understood the meaning of “about” in association with a specific context. In some instances, the term “about” refers to a particular value+/−5% (e.g., +/−3% or +/−2%).

In some instances, the topical formulation may further comprise one or more carriers or excipients, including one or more of viscosity increasing agents (e.g., about 1.0-10%), one or more ointment bases (e.g., one or more cream base) which may range from about 5-30%, one or more antimicrobial preservative (e.g., about 0.005-0.2% by weight), one or more emulsifying agents (about 0.5-10% by weight) or a combination thereof. These components may be dissolved or disbursed in a suitable solvent.

A “viscosity increasing agent” is an agent that is used to thicken a formulation. Exemplary viscosity increasing agents may include, for example, cetostearyl alcohol, cholesterol, stearyl alcohol, chlorocresol, white wax, stearic acid, cetyl alcohol, or a combination thereof. The viscosity increasing agent may be present in the topical formation at a concentration of about 1.0-10% (w/w). For example, the topical formulation may comprise about 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, or 9.5-10% (w/w) of the viscosity increasing agent. Alternatively, the topical formulation may comprise about 1-5%, 2.5-7.5%, or 5-10% (w/w) of the viscosity increasing agent.

An “ointment base” can be any semisolid preparation or vehicle into which an active agent may be incorporated. Exemplary ointment bases include, but are not limited to, oleaginous ointment bases (e.g., white petrolatum or white ointment), absorption ointment bases (e.g., hydrophilic petrolatum, anhydrous lanolin, Aquabase™, Aquaphor®, and Polysorb®), water/oil emulsion ointment bases (e.g., cold cream, hydrous lanolin, rose water ointment, Hydrocream™, Eucerin®, and Nivea®), oil/water emulsion ointment bases (e.g., hydrophilic ointments, Dermabase™, Velvachol®, and Unibase®), and water-miscible ointment bases (e.g., polyethylene glycol (PEG) ointment and Polybase™). Ointment bases may be pharmacologically inert but can entrap water in order to provide an emollient protective film. In a specific embodiment, the ointment base may be any petrolatum compound (e.g., petrolatum, white petrolatum, white soft paraffin, liquid petrolatum, liquid paraffin). In a further specific embodiment, the ointment base is white petrolatum (CAS number 8009-03-8). The ointment base may be present in the topical formation at a concentration of about 5-30% (w/w), e.g., 10-30% (w/w). For example, the topical formulation may comprise about 5-25%, 5-20%, 5-15%, 5-15%, 10-15%, 15-20%, 20-25%, or 25-30% (w/w) of the ointment base. Specifically, the topical formulation may comprise about 5, 6, 7, 8, 9, 10, 11, 12, 13, 14, 15, 16, 17, 18, 19, 20, 21, 22, 23, 24, 25, 26, 27, 28, 29, or 30 percent (w/w) of the ointment base.

In some embodiments, the “ointment base” described herein contains less than 20% water and volatiles, and more than 50% hydrocarbons, waxes, or polyols as the vehicle.

In some embodiments, the “ointment base” described herein is a “cream base,” which contains more than 20% water and volatiles and/or typically contain less than 50% hydrocarbons, waxes, or polyols as the vehicle for the drug substance. The cream base can be a multiphase preparation containing a lipophilic phase and an aqueous phase. In some instances, the cream base is a lipophilic cream base, which has a lipophilic phase as the continuous phase. Such a cream base usually contains water-in-oil emulsifying agents such as wool alcohols, sorbitan esters and monoglycerides. In other instances, the cream base is a hydrophilic cream base, which has an aqueous phase as the continuous phase. Such a cream base typically contains oil-in-water emulsifying agents such as sodium or trolamine soaps, sulfated fatty alcohols, polysorbates and polyoxyl fatty acid and fatty alcohol esters, which may be in combination with water-in-oil emulsifying agents, if needed.

An “antimicrobial preservative” can be any compound capable of destroying microbes, prevent the multiplication or growth of microbes, or prevent the pathogenic action of microbes. Exemplary antimicrobial preservatives include, but are not limited to, a paraben compound (an ester of para-hydroxybenzoic acid; e.g., paraben, methylparaben, ethylparaben, propylparaben, butylparaben, heptylparaben, benzylparaben, isobutylparaben, isopropylparaben, benzylparaben, or their sodium salts), benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. The antimicrobial preservative may be present in the topical formation at a concentration of about 0.005-0.2%, e.g., about 0.01-0.2% (w/w). For example, the topical formulation may comprise about 0.005-0.01%, 0.01-0.05%, 0.05-0.1%, 0.1-0.15%, or 0.15-0.2% (w/w) of the antimicrobial preservative. Specifically, the topical formulation may comprise about 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 percent (w/w) of the antimicrobial preservative.

An “emulsifying agent” is a compound or substance which acts as a stabilizer for a mixture of two or more liquids that are normally immiscible (unmixable or unblendable). Exemplary emulsifying agents may include, but are not limited to, natural emulsifying agents (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [Tween® 20], polyoxyethylene sorbitan [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, and sorbitan monooleate [Span® 80]), polyoxyethylene esters (e.g., polyoxyethylene monostearate [Myrj® 45], polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers (e.g., polyoxyethylene lauryl ether [Brij® 30]), and poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, and docusate sodium, and/or combinations thereof. The emulsifying agent may be present in the topical formation at a concentration of about 0.5-10% (w/w), e.g., 0.5-6% (w/w). For example, the topical formulation may comprise about 0.5-1%, 1-1.5%, 1.5-2%, 2-2.5%, 2.5-3%, 3-3.5%, 3.5-4%, 4-4.5%, 4.5-5%, 5-5.5%, 5.5-6%, 5-10%, 6-10%, or 8-10% (w/w) of the emulsifying agent. Specifically, the topical formulation may comprise about 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 percent (w/w) of the emulsifying agent.

The topical formulation of the invention may further contain one or more solvents (e.g., non-water solvents or water). Exemplary non-water solvents may include, but are not limited to, any known solvent including propylene glycol, glycol, and mixtures thereof. The non-water solvent may be present in the topical formation at a concentration of about 2-65% (w/w). For example, the topical formulation may comprise about 2-15%, 15-30%, 30-45%, or 45-65% (w/w) of the solvent. In some embodiments, the topical formulation of the invention may also contain water.

In some embodiments, the topical formulation of the invention may further comprise one or more emollients, fragrances, or pigments. The topical formula may also be used in conjunction with a wound dressing (e.g., bandage with adhesive, plaster patch and the like). (e.g., cyclohexane, n-hexane, n-decane, i-octane, octane, butyl ether, carbon tetrachloride, triethyl amine, i-propyl ether, toluene, p-xylene, t-butyl methyl ether, benzene, benzyl ether, dichloromethane, methylene chloride, chloroform, dichloroethane, ethylene dichloride, 1-butanol, i-butyl alcohol, tetrahydrofuran, ethyl acetate, 1-propanol, 2-propanol, methyl acetate, cyclohexanone, methyl ethyl ketone (MEK), nitrobenzene, benzonitrile, 1,4-dioxane, or p-dioxane).

Additional information regarding topical formulations can be found, e.g., in U.S. Pat. No. 10,758,584, the relevant disclosures of which are incorporated by reference for the subject matter and purpose referenced herein.

The topical formulation disclosed herein may be in any suitable format. Examples include, but are not limited to, cream, dressing, ointment, lotion, paste, and gel. In some instances, the topical formulation may be placed on a device (e.g., a patch) for applying to a site where skin disorders occur.

### II. Methods for Alleviating Radiation-Induced Skin Disorders

To practice the method disclosed herein, an effective amount of the composition (e.g., the pharmaceutical composition) described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution.

In some instances, a topical formation as described herein can be used for alleviating radiation-induced skin disorders in a subject in need of the treatment. Exemplary radiation-induced skin disorders include, but are not limited to, radiation dermatitis (e.g., acute or chronic), radiation-induced mucositis, radiation-induced lichen planus, radiation-induced vaginitis, radiation-induced skin inflammation, radiation-induced skin damage, or a combination thereof.

The topical formulation may be applied to a damage site following a suitable dosage and treatment regimen. The dosage and administration regimen for the described method will depend on the nature and condition of the symptoms being treated, the age and condition of the patient, and any prior or concurrent therapy. In some instances, the topical formulation can be applied once every week, once every other day, once daily, twice daily, three times daily, or four time daily for a suitable period of time. The treatment may be terminated when the radiation-induced skin disorder is recovered. When necessary, the treatment may resume, for example, if one or more symptoms of skin disorder recurs.

The term “radiation-induced skin disorder” refers to any skin disorder associated with exposure to radiation. Examples include radiation dermatitis (e.g., acute or chronic), radiation-induced mucositis, radiation-induced lichen planus, radiation-induced vaginitis, radiation-induced skin inflammation, radiation-induced skin damage, or a combination thereof.

The term “radiation dermatitis,” also known as radiation burn, refers to dermatitis (e.g., skin irritation) associated with radiation. In some instances, radiation dermatitis is associated a radiotherapy received by a subject, for example, a human cancer patient receiving an anti-cancer radiotherapy (e.g., an X-ray therapy). Common symptoms associated with radiation dermatitis include redness, itching, flaking, peeling, soreness, moistness, blistering, pigmentation changes, fibrosis, scarring of connective tissue and development of ulcers. Gradings of radiation dermatitis are provided in Table 1 below, following the National Cancer Institute Common Terminology Criteria for Adverse Events (CTCAE) v5.0 guidance:

TABLE 1
NCI CTCAE v5.0 Radiation Dermatitis Grading

| Grade | Symptoms |
|---|---|
| 1 | Faint erythema or dry desquamation |
| 2 | Moderate to brisk erythema; patchy moist desquamation; |
| | moistly confined to skin folds and creases; moderate edema |
| 3 | Moist desquamation in areas other than skin folds and creases; |
| | bleeding induced by minor trauma or abrasion |
| 4 | Life-threatening consequences; skin necrosis or ulceration o = f |
| | full thickness dermis; spontaneous bleeding from involved site; |
| | skin graft indicated |
| 5 | Death |

The subject to be treated by the composition disclosed herein (e.g., any of the topical formulations disclosed herein) can be a human or a non-human mammal. In some embodiments, the subject is a human patient having a radiation-induced skin disorder (e.g., radiation dermatitis), suspected of having the skin disorder, or at risk of developing the skin disorder. In some instances, the subject may be a human patient (e.g., a human cancer patient) who has received or is receiving a radiotherapy. In some examples, treatment with any of the compositions disclosed herein for the radiation-induced skin disorder and a radiotherapy may be performed concurrently to a subject in need of the treatments.

A subject having the radiation-induced skin disorder as those disclosed herein can be identified by routine medical examinations. In some instances, the subject having radiation dermatitis may show one or more of the symptoms disclosed herein. In other instances, the subject having radiation dermatitis may suffer skin cell damage at a deep skin layer with little or no surface wounds. A subject suspected of having radiation dermatitis might show one or more symptoms of the disease/disorder. A subject at risk for radiation dermatitis can be a subject having one or more of the risk factors for that disease/disorder (e.g., a human patient who will receive a radiotherapy or who is at risk of being exposed to radiation).

As used herein, “an effective amount” refers to the amount of each active agent required to confer therapeutic effect on the subject, either alone or in combination with one or more other active agents. Determination of whether an amount of the composition achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of the composition may be appropriate. Various formulations and devices for achieving sustained release are known in the art.

In one example, dosages for a composition as described herein may be determined empirically in individuals who have been given one or more administration(s) of the composition. Individuals are given incremental dosages of the agonist. To assess efficacy of the agonist, an indicator of the disease/disorder can be followed.

Generally, for administration of any of the compositions described herein, an initial candidate dosage can be given to a subject. For repeated administrations over several days or longer, depending on the condition, the treatment is sustained until a desired suppression of symptoms occurs or until sufficient therapeutic levels are achieved to alleviate a target disease or disorder, or a symptom thereof. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of the composition as described herein will depend on the specific active agent contained therein, the type and severity of the disease/disorder, whether the composition is administered for preventive or therapeutic purposes, previous therapy, the patient's clinical history and response to treatment, and the discretion of the attending physician. Typically, the clinician will administer the composition, until a dosage is reached that achieves the desired result. In some embodiments, the desired result is a reduction of disease severity (e.g., represented by a disease score). Methods of determining whether a dosage resulted in the desired result would be evident to one of skill in the art. Administration of any of the compositions disclosed herein can be continuous or intermittent, depending, for example, upon the recipient's physiological condition, whether the purpose of the administration is therapeutic or prophylactic, and other factors known to skilled practitioners. The administration of the composition may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

The particular dosage regimen, i.e., dose, timing and repetition, used in the method described herein will depend on the particular subject and that subject's medical history.

In some embodiments, any of the compositions disclosed herein may be co-used with one or more additional therapeutic agents for treating the target disease. In some instances, the additional therapeutic agents may serve to enhance and/or complement the effectiveness of the composition disclosed herein.

Treatment efficacy for a target disease/disorder can be assessed by methods well-known in the art.

### III. Kits for Use in Alleviation of Radiation-Induced Skin Disorder

The present disclosure also provides kits for use in alleviating radiation-induced skin disorders such as radiation dermatitis, radiation-induced mucositis, radiation-induced lichen planus, radiation-induced vaginitis, radiation-induced skin inflammation, radiation-induced skin damage, or a combination thereof. Such kits may include one or more containers comprising any of the compositions (e.g., a topical formulation) as described herein, which comprises a Plectranthus amboinicus extract, a Centella asiatica extract, a combination of the Plectranthus amboinicus extract and the Centella asiatica extract, or one or more active agents thereof such as those disclosed herein.

In some embodiments, the kit may comprise instructions for use in accordance with any of the methods described herein. The included instructions may comprise a description of administration of the composition (e.g., a topical formulation) to alleviate radiation-induced skin disorders such as those disclosed herein according to any of the methods described herein. The kit may further comprise a description of selecting an individual suitable for treatment based on identifying whether that individual has the skin disorder in need of treatment.

The instructions relating to the use of the composition (e.g., a topical formulation) generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for alleviating radiation-induced skin disorders as disclosed herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. At least one active agent in the composition is an active agent as disclosed herein, which may be contained in a Plectranthus amboinicus extract, a Centella asiatica extract, or a combination of a Plectranthus amboinicus extract and a Centella asiatica extract.

Kits may optionally provide additional components such as interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Alleviation of Radiation Dermatitis with Plectranthus Amboinicus (PA)

Extract, Optionally in Combination with Centella Asiatica (CA) Extract This example explores treatment efficacy of radiation dermatitis using (a) ON101 cream, which comprises a Plectranthus amboinicus (PA) extract and a Centella asiatica extract; and (b) PA-F4 cream, which comprises the Plectranthus amboinicus (PA) extract. A topical corticosteroid cream (Betamethasone) is used as a positive control.

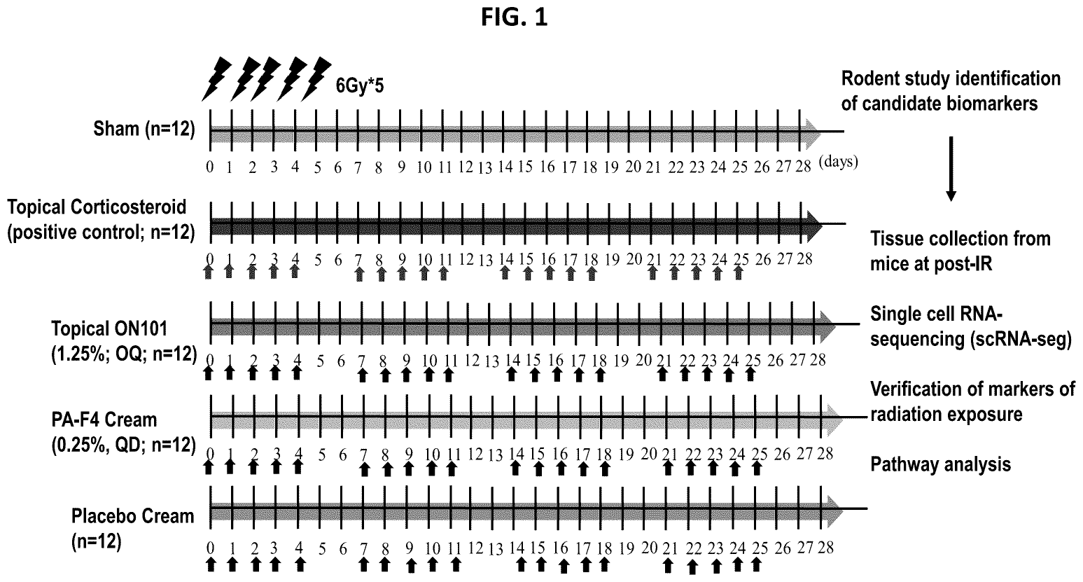

FIG. 1 depicts the overall experimental design of a mice radiation study to investigate therapeutic effects of ON101 cream and PA-F4 cream. Briefly, mice were shaved to remove back hair, anesthetized and then received one dose of 6.5 Gy daily at shaved sites for 5 days (Day 0 to Day 4). The ON101 cream, PA-F4 cream, or the topical corticosteroid cream (control) was applied to the irradiated sites. Radiation-induced dermatitis were evaluated during Day 8 to Day 28 once every 2 days. 1 ml of the cream was applied to each of 5 cm² shaved areas once per day for five days, followed by two-day resting period. This cycle was repeated for 4 times. See FIG. 1. During this period, levels of radiation dermatitis were determined following the definitions provided in Table 2 below:

TABLE 2
Definitions of Radiation Dermatitis Scores

| | | Correspondence |
|---|---|---|
| Score | Symptoms | to CTCAE |
| 0 | None | |
| 0.5 | Dryness | |
| 1 | Dry desquamation | CTCAE Grade 1 |
| 2 | Early moist desquamation | CTCAE Grade 2 |
| 3 | Moist desquamation in about 30% of areas | CTCAE Grade 3 |
| 4 | Moist desquamation in more than 50% of areas | |
| 5 | Skin necrosis and ulceration | |

Skin tissue samples were collected for analysis of biomarkers of radiation exposure, via, e.g., single cell RNA-sequencing (scRNA-seq).

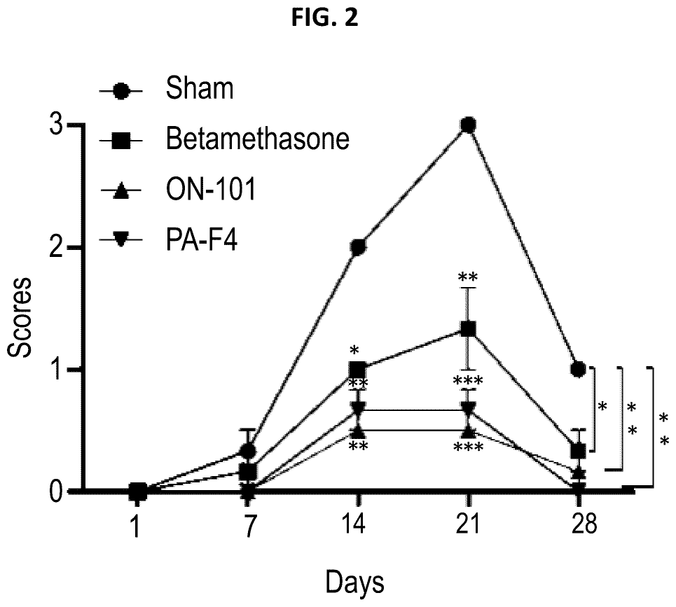

As shown in FIG. 2, both the ON101 cream and the PA-F4 cream showed significant reduction of radiation dermatitis scores and the therapeutic effects are better than that of the control cream.

### Example 2: Treatment Efficacy of Radiation-Induced Skin Damage by PA Exact, Optionally in Combination with CA Extract

This example evaluates treatment efficacy of radiation-induced skin damage by the PA extract, either taken alone or in combination with the CA extract as disclosed herein. The treatment process is described in Example 1 above. See also FIG. 1.

(i) Thickness of Epiderma

Thickness of epiderma in mice treated by the ON101 cream, PA-F4 cream, corticosteroid (positive control), a placebo cream (negative control), and mice with no treatment (blank control) was measured by hematoxylin and Eosin (H&E) staining. The tissue sections obtained from the mice were submerged in distilled water and alum haematoxylin was applied for staining nuclei. The tissue sections were then cleaned by rinsing them under running tap water. 0.3% acid alcohol was used for differentiation. The tissue section were rinsed again under running tap water and then submerged in Scott's tap water for further washing, followed by rinsing with tap water. The tissue sections were then stained in an esosin solution for 2 minutes and then dehydrated, cleared, and mounted.

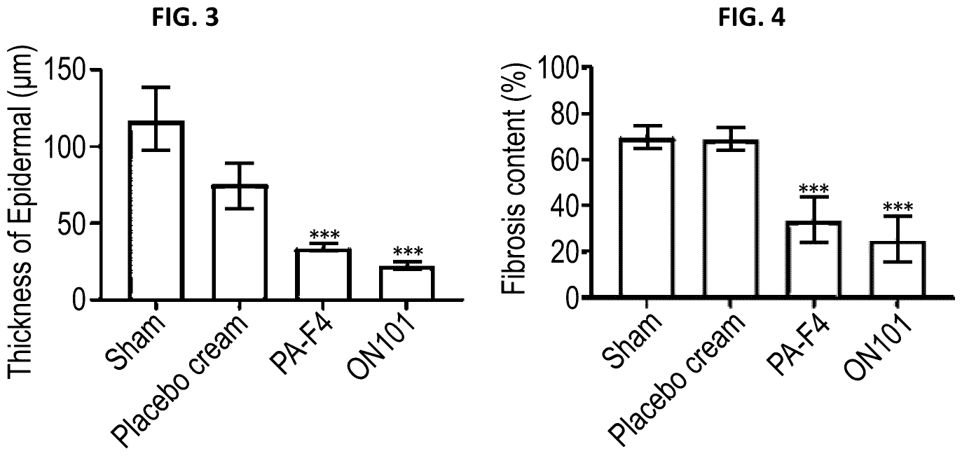

The results show that both ON101 and PA-F4 reduced the thickness of epiderma with ON101 showing a better effect. FIG. 3.

(ii) Fibrosis Contents

Fibrosis contents of the mice at the radiation-treated sites were analyzed by Masson staining. Masson's trichrome stain is a widely utilized method in histology for the identification and differentiation of collagen fibers and muscle fibers in tissue sections. This staining technique is particularly valuable in studying various pathologies affecting organs. To perform Masson's trichrome staining, the following steps were carried out.

Initially, the skin samples obtained from the mice of various treatment groups were subject to deparaffinization and rehydration via sequentially passing through 100% alcohol, 95% alcohol, and 70% alcohol. This process ensures the removal of the paraffin wax and allows the sample to be properly prepared for staining. Subsequently, the samples were thoroughly washed in distilled water to eliminate any residual chemicals or debris, ensuring a clean surface for the staining.

The treated skin samples were then incubated in the Weigert's iron hematoxylin working solution for 10 minutes. This step facilitates the visualization of cell nuclei. Following the staining step, the samples were rinsed in running warm tap water for approximately 10 minutes. This rinsing process effectively removes excess stain, enhancing the clarity of the stained sections. After rinsing, the samples were once again washed in distilled water to ensure the removal of any remaining traces of the staining solution.

Finally, the samples were stained using a Biebrich scarlet-acid fuchsin solution for a period of 10-15 minutes. This staining solution imparts distinct coloration to collagen fibers, allowing for their differentiation from other tissue components.

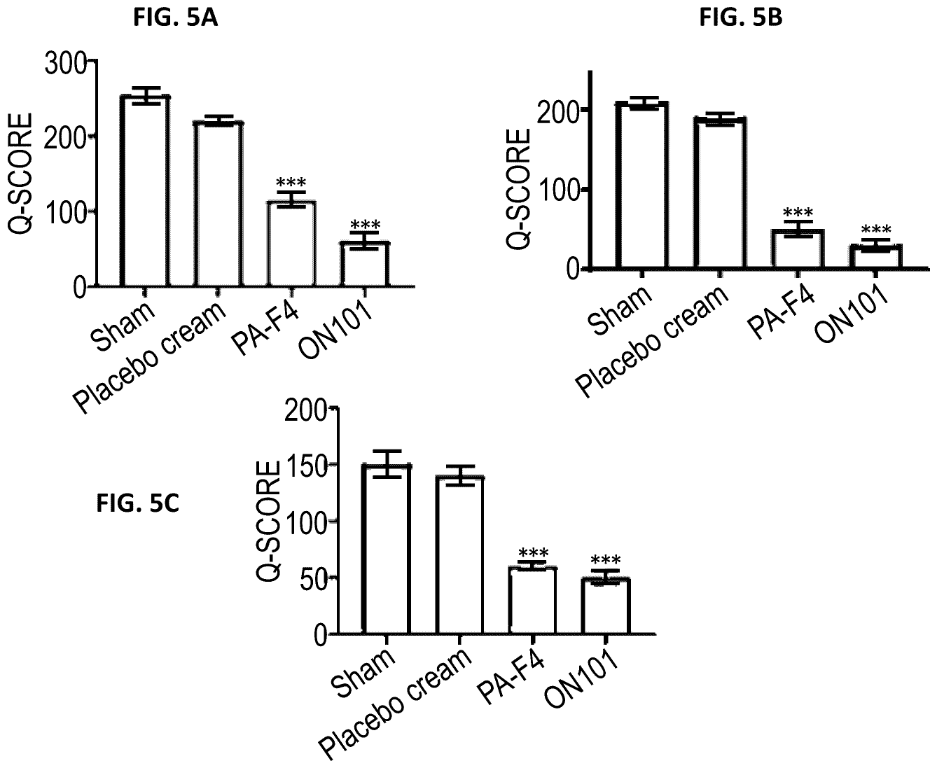

As shown in FIG. 4, skin samples from mice treated by the PA-F4 cream and ON101 cream showed reduced fibrosis contents as relative to mice treated by the placebo cream or untreated mice.

(iii) Immunohistochemical Analysis

Immunohistochemical analysis was performed to investigate the pathways associated with oxidative stress, radiation damage, and inflammation in mice treated with the various creams as described above. This experiment involved categorizing mouse skin tissue samples obtained from mice treated with ON101, PA-F4, sham, or placebo cream. Immunostaining analysis was conducted for examining the levels of NLRP3, rH2AX, and IL1B via the immunohistochemical (IHC) process as follows.

First, the slides were washed three times with PBS. Subsequently, 600 μl of peroxidase-block solution was added to the slides, which were then incubated for 10 minutes. After another round of PBS wash, the slides were blocked in 2% skim milk for 15 minutes. Then, mouse anti-NLRP3, rH2AX, or IL1B (diluted 1:500) was applied to the slides, which were incubated at 4° C. for 20 hours. Following three washes, Dako HRP-labelled X anti-mouse (1:5000) was added, and the slides were incubated for 30 minutes before undergoing another three washes. Next, a substrate containing chromogen (1 drop of chromogen to 1 ml substrate) was added to the slides and incubated for 1-15 minutes. The slides were washed thrice with PBS and subsequently counterstained with Mayer's hematoxylin for 12 seconds. After rinsing with tap water, the slides were left to dry.

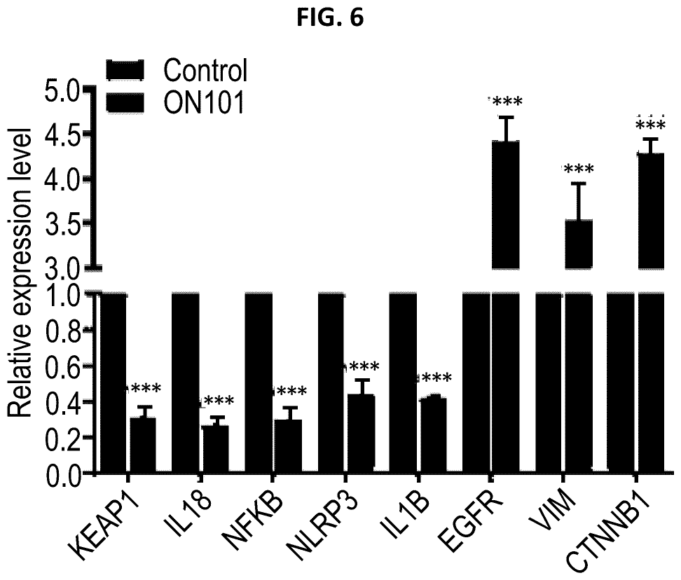

The levels of NLRP3, rH2AX, and IL1B in mouse skin samples were summarized in FIGS. 5A-5C, respectively. Mice treated by the ON101 cream and the PA-FA cream exhibited significantly lower levels of NLRP3, rH2AX, and IL1B in skin tissues as compared with untreated mice or mice treated with the placebo cream.

(iv) Potential Pathway for ON101 Protection Against Radiation Dermatitis

In this study, RNA-sequencing analysis was conducted on mouse tissue samples affected by radiation dermatitis to explore the molecular changes occurring in the skin tissues during radiation dermatitis and to investigate potential pathways through which ON101 confers its protective effects.

RNA transcripts collected from mouse skin samples were analyzed using the high-throughput RNA-sequencing technology. This technique allows for a comprehensive view of the gene expression changes occurring in the skin during radiation dermatitis, in the presence or absence of ON101 treatment. The results of the RNA-sequencing analysis could be used to identify differentially expressed genes and pathways that are associated with the development and progression of radiation dermatitis. By comparing the gene expression profiles of the samples obtained from ON101-treated mice to samples obtained from untreated mice, specific genes and pathways regulated by ON101 treatment could be deciphered.

Further, RT-qPCR analysis were performed using the miDETECT A Track™ RNA qRT-PCR Starter Kit (RIBOBIO, Guangzhou, Taiwan) to investigate levels of RNA transcripts. This process involved converting miRNAs into complementary DNA (cDNA) using the PrimeScript® 1st Strand cDNA Synthesis Kit (TaKaRa, Taipei, Taiwan). To quantify gene expression levels, qRT-PCR was carried out utilizing the SYBR Premix Ex Taq II kit (Takara, Taipei, Taiwan). The qRT-PCR analysis was performed in the CFX96 Real-Time PCR Detection System (Bio-Rad, Hercules, CA). By utilizing these molecular biology tassays and kits, the miRNA and gene expression levels related to the Nrf2-rH2AX axis were measured and analyzed.

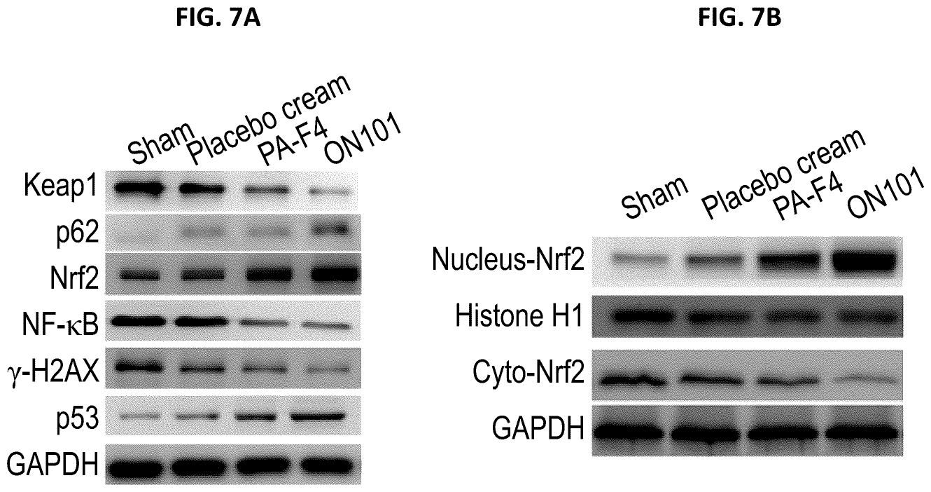

Results from this study show that genes such as KEAP1, IL18, NK-κB, NLRP3, EGFR, VIM, and CTNNB1 were differentially expressed in samples from ON101-treated mice as compared to samples from untreated mice. More specifically, ON101 reduced expression levels of KEAP1, IL18, NK-κB, NLRP3, and IL1B and enhanced expression levels of EGFR, VIM, and CTNNB1. FIG. 6. The results suggest that ON101 could mitigate the development and severity of radiation dermatitis by targeting Keap1, a protein known to play a crucial role in oxidative stress response and inflammation.

(v) Activation of p62-KEAP1-NRF2 Pathway and Promoted Nrf2 Nuclear Translocation in Radiation Dermatitis Mice

In this study, ON101 and PA-F4 were found to activate the p62-KEAP1-NRF2 pathway and promoted Nrf2 nuclear translocation in radiation dermatitis mice.

The protein lysates prepared from skin samples collected from mice treated by ON101, PA-F4, or the placebo cream, as well as from untreated mice, were heat treated to prepare them for analysis. To perform immunoblotting, a 5% skimmed milk solution in Tris-buffered saline with Tween® 20 (TBST) was used, following the conventional immunoblotting practices. Briefly, proteins in the protein lysate samples were transferred to a polyvinylidene fluoride (PVDF) membrane, which was then incubated with a primary antibody specific to a target protein (Keap1, p62, Nrf2, NFKB, r-H2AX, P53, GAPDH, Nucleus-Nrf2, Histone H1, Cyto-Nrf2) overnight at 4° C. The membrane was then washed several times with TBST and then incubated with a secondary antibody labeled with horseradish peroxidase (HRP) at room temperature for 1 hour. After being washed by TBST, levels of the various target proteins on the membrane were examined by enhanced chemiluminescence (ECL). Finally, a BioSpectrum Imaging System (UVP, Upland, CA, USA) was employed to capture and analyze the generated chemiluminescent signals.

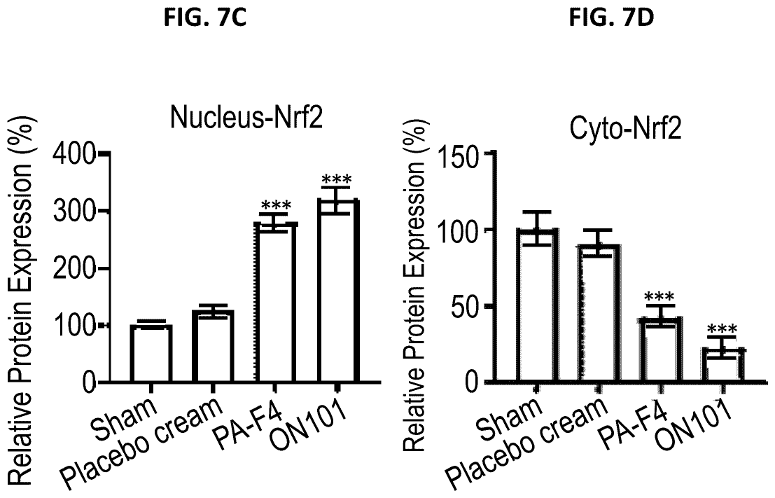

The levels of the various target proteins examined are shown in FIGS. 7A and 7B. As shown in FIGS. 7C and 7D, samples from PA-F4-treated or ON101-treated mice showed higher levels of nuclease Nrf2 and reduced levels of cytoplasmic Nrf2, as compared to samples from mice treated with the placebo cream or untreated mice, indicating that PA-F4 and ON101 can enhance nuclear-cytoplasmic translocation of Nrf2.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for alleviating a radiation-induced skin disorder, comprising administering to a subject in need thereof an effective amount of a composition comprising salvigenin and a carrier, optionally wherein the composition is a pharmaceutical composition which further comprises a pharmaceutically acceptable carrier.

2. The method of claim 1, wherein the composition further comprises cirsimaritin, rosmarinic acid, carvacrol, or a combination thereof.

3. The method of claim 1, wherein the composition comprises a Plectranthus amboinicus (PA) extract.

4. The method of claim 3, wherein the PA extract is prepared by a process comprising:
(i) mixing a part of PA with an extracting solution to produce a first PA extract,
(ii) filtrating and concentrating the first PA extract to produce a concentrated PA extract;
(iii) contacting the concentrated PA extract onto a hydrophobic interaction chromatography resin, and
(iv) eluting the column with an eluent solution to product the PA extract.

5. The method of claim 4, wherein the part of PA in step (i) is an above-ground part.

6. The method of claim 4, wherein the extracting solution comprises a solvent having a polarity index of about 2.9 to 6.6.

7. The method of claim 6, wherein the extracting solution is acetone, butyl methyl ether, ethanol, ethyl acetate, isopropyl alcohol, methanol, or a mixture thereof.

8. The method of claim 4, wherein the eluent solution comprises a solvent having a polarity index of about 2.1-5.4.

9. The method of claim 8, wherein the eluent solution comprises a mixture of at least two solvents selected from the group consisting of acetone, ethanol, ethyl acetate, and hexane.

10. The method of claim 1, wherein the composition further comprises asiaticoside.

11. The method of claim 10, wherein the composition further comprises pharmaceutical a Centella asiatica (CA) extract, which comprises the asiaticoside.

12. The method of claim 1, wherein the subject is a human patient having the radiation-induced skin disorder.

13. The method of claim 1, wherein the composition is a topical formulation.

14. The method of claim 13, wherein the topical formulation is in a form of cream, gel, dressing, spray formulation, ointment, paste, patch, or lotion.

15. The method of claim 1, wherein the composition is applied to a site where the radiation-induced skin disorder occurs.

16. The method of claim 1, wherein the subject is receiving a radiotherapy.

17. The method of claim 1, wherein the subject is a cancer patient.

18. The method of claim 1, wherein the composition is administered to the subject once every three days to six times per day.

19. The method of claim 1, wherein the radiation-induced skin disorder comprises radiation dermatitis, radiation-induced mucositis, radiation-induced lichen planus, radiation-induced vaginitis, radiation-induced skin inflammation, radiation-induced skin damage, or a combination thereof.
